(19) United States
(12) Patent Application Publication
(10) Pub. No.: US 2021/0393921 A1
(43) Pub. Date: Dec. 23, 2021

(54) PROBE ASSEMBLY TO REPOSITION A CATHETER

(71) Applicant: Becton, Dickinson and Company, Franklin Lakes, NJ (US)

(72) Inventors: Jonathan Karl Burkholz, Salt Lake City, UT (US); Curtis H. Blanchard, Riverton, UT (US); John Lackey, West Valley City, UT (US); Weston F. Harding, Lehi, UT (US)

(21) Appl. No.: 17/330,224

(22) Filed: May 25, 2021

Related U.S. Application Data
(60) Provisional application No. 63/041,548, filed on Jun. 19, 2020.

(51) Int. Cl. A61M 25/01; A61M 25/00; A61B 5/15

(52) U.S. Cl. CPC A61M 25/0138; A61B 5/150992; A61M 25/0017

(57) ABSTRACT

A probe can be configured with a shaped portion that can lift, advance, retract, or swivel a distal end of a catheter to thereby reposition the catheter within a patient's vasculature. This repositioning can move the catheter relative to the wall or other anatomy of the vasculature and relative to any obstructions such as a thrombus that may have formed. By repositioning the catheter, the probe prolongs the patency of the catheter including facilitating the collection of a blood sample through a long-dwelling catheter.

### RELATED APPLICATIONS

This application claims benefit of U.S. Provisional Patent Application No. 63/041,548, filed on Jun. 19, 2020, entitled INTRAVENOUS CATHETER DEVICE WITH PROBE FOR REPOSITIONING THE CATHETER, which is incorporated herein in its entirety.

### BACKGROUND

Intravenous (IV) catheter devices are commonly used for a variety of infusion therapies. For example, an IV catheter device may be used for infusing fluids, such as normal saline solution, various medicaments, and total parenteral nutrition, into a patient. IV catheter devices may also be used for withdrawing blood from the patient.

A common type of IV catheter device includes a catheter that is “over-the-needle.” As its name implies, the catheter that is over-the-needle may be mounted over a needle having a sharp distal tip. The catheter and the needle may be assembled so that the distal tip of the needle extends beyond the distal tip of the catheter with the bevel of the needle facing up away from skin of the patient. The catheter and needle are generally inserted at a shallow angle through the skin into the vasculature of the patient.

When IV catheter devices are maintained within the patient's vasculature, they are likely to become occluded. Once an IV catheter device is occluded, it may no longer be possible to use the IV catheter device to infuse fluids or withdraw blood. In such cases, the IV catheter device may be replaced. Yet, replacing an IV catheter device is burdensome for the patient and increases costs. To address such issues, some devices have been developed that can be inserted through the indwelling catheter of the IV catheter device to remove the occlusion. For example, some devices employ rigid tubing that can be inserted through the catheter and distally beyond the catheter's distal opening. With the rigid tubing inserted in this manner, such devices can obtain a blood sample through the rigid tubing even if the catheter had become occluded. In other words, the rigid tubing is employed to physically pass through any occlusion that may have formed in or around the catheter's distal opening and forms a separate fluid pathway from the catheter for collecting the blood sample.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to probe assemblies configured to reposition a distal end of a catheter while the catheter remains inserted in a patient's vasculature, as well as related methods and IV catheter devices. In some embodiments, an IV catheter device may include a catheter adapter, a catheter that extends distally from the catheter adapter and a probe assembly that couples to the catheter adapter. The probe assembly may include a probe that selectively extends into the catheter. The probe may have a shaped portion for causing a distal end of the catheter to be repositioned as the probe is selectively extended into the catheter. The probe assembly may be integrated into or selectively coupled to the catheter adapter. The probe may be a wire or a tube in some embodiments.

The probe can be configured with a shaped portion that can lift, advance, retract, or swivel the distal end of the catheter to thereby reposition the catheter within the patient's vasculature. This repositioning can move the catheter relative to the wall or other anatomy of the vasculature and relative to any obstructions such as a thrombus that may have formed. By repositioning the catheter, the probe prolongs the patency of the catheter including facilitating the collection of a blood sample through a long-dwelling catheter.

In some embodiments, the probe assembly may include a probe actuator by which the probe is selectively extended into the catheter. In some embodiments, the probe actuator may be configured to advance axially and/or to rotate to thereby cause the probe to advance axially and/or rotate within the catheter respectively.

In some embodiments, the probe assembly may include a probe housing within which the probe is housed, and a probe actuator positioned at least partially outside of the probe housing. The probe actuator may interface with the probe to cause the probe to be selectively advanced out from the probe housing and into the catheter. The probe actuator may also interface with the probe to cause the probe to rotate within the catheter.

In some embodiments, the probe may have a proximal portion and a distal portion, and the shaped portion may be positioned between the proximal portion and the distal portion. In some embodiments, the shaped portion may encompass the distal portion. In some embodiments, the distal portion may form a coil. In some embodiments, the shaped portion may have a v shape or a w shape or may form a spiral or other shape.

In some embodiments, the probe may include a proximal portion and the shaped portion may be distal to the proximal portion. The shaped portion may include a first length that deviates from a longitudinal axis of the proximal portion by a first angle and a second length that deviates from the longitudinal axis of the proximal portion by a second angle different from the first angle.

In some embodiments, a probe assembly for use with a catheter of an intravenous catheter device may include a probe housing, a probe actuator coupled to the probe housing and a probe that is housed within the probe housing. The probe actuator may be configured to selectively advance the probe from the probe housing and into the catheter of the intravenous catheter device. The probe may include a proximal portion, a distal end and a shaped portion positioned between the proximal portion and the distal end. The shaped portion may be configured to reposition a distal end of the catheter as the probe is selectively advanced within the catheter.

In some embodiments, the shaped portion may include a first length that deviates from a longitudinal axis of the proximal portion by a first angle and a second length that deviates from the longitudinal axis of the proximal portion by a second angle different from the first angle. In some embodiments, the probe actuator may be configured to selectively rotate the probe within the catheter. In some embodiments, the shaped portion of the probe may encompass the distal end of the obturator.

In some embodiments, an intravenous catheter device may include a catheter adapter, a catheter that extends distally from the catheter adapter and a probe assembly that couples to the catheter adapter. The probe assembly may include a probe actuator and a probe having a proximal end coupled to the probe actuator and a distal end. The probe may have a shaped portion positioned towards the distal end. The shaped portion may be configured to cause a distal end of the catheter to be repositioned while the probe is extended into the distal end of the catheter. In some embodiments, the probe actuator may be configured to slide and rotate the shaped portion within the catheter.

It is to be understood that both the foregoing general description and the following detailed description are examples and explanatory and are not restrictive of the invention, as claimed. It is to be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present disclosure. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the accompanying drawings in which:

FIG. 1 illustrates an example of an IV catheter device that includes a probe for repositioning the catheter in accordance with some embodiments;

FIGS. 2A and 2B illustrate an example of a probe assembly that may be used with an IV catheter device in some embodiments;

FIGS. 3A-3I illustrate a number of examples of a probe that is configured to reposition the distal end of a catheter while the catheter remains in a patient's vasculature in accordance with some embodiments;

FIGS. 4A-4C illustrate an example of how the distal end of a catheter can be repositioned while the catheter remains in a patient's vasculature using a probe that is configured in accordance with some embodiments;

FIG. 5 illustrates an example of a probe that is configured to rotate to cause a catheter to be repositioned within a patient's vasculature in accordance with some embodiments; and

FIGS. 6A-6D illustrate another example of how the distal end of a catheter can be repositioned while the catheter remains in a patient's vasculature using a probe that is configured in accordance with one or more embodiments.

### DESCRIPTION OF EMBODIMENTS

An IV catheter device that may be employed in some embodiments may include a catheter adapter from which a catheter distally extends and one or more ports or connectors for attaching other devices to the catheter adapter. Such devices may be attached to the catheter adapter before, during or after insertion of the catheter into a patient's vasculature and can include a needle assembly, a blood collection set, an infusion assembly, any embodiment of a probe assembly described herein, etc. Accordingly, embodiments of the present disclosure should not be limited to any particular configuration of an IV catheter device or to the specific examples of IV catheter devices used herein.

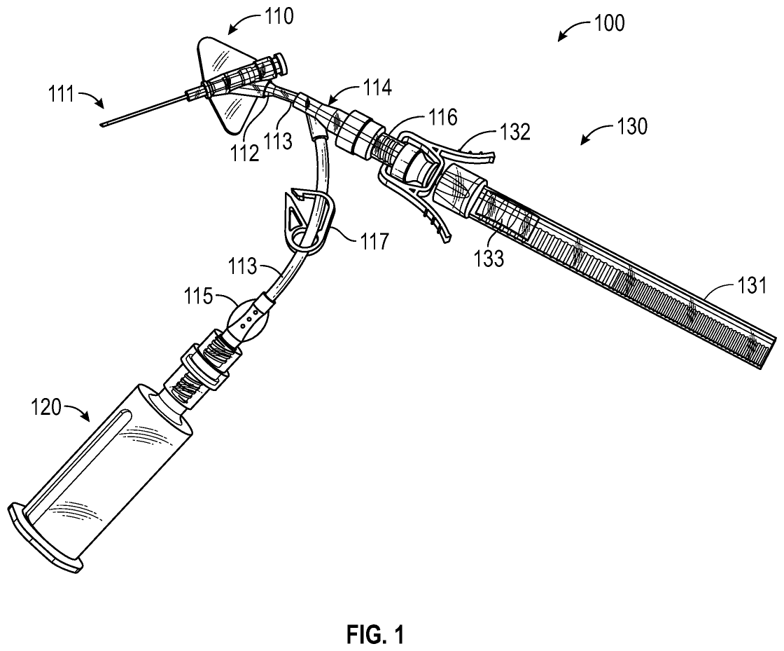

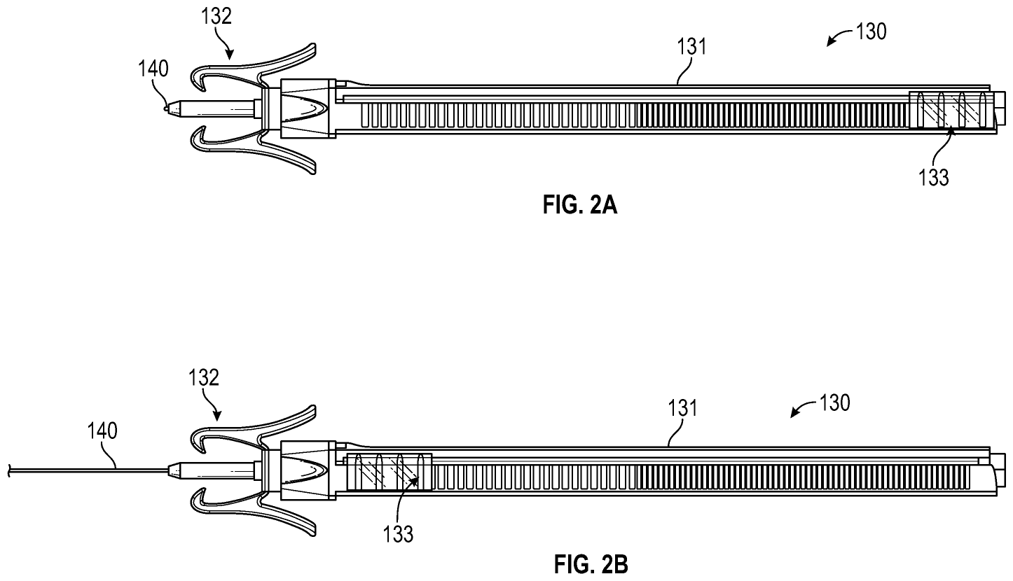

FIG. 1 provides an example of an IV catheter device 100 that is configured in accordance with some embodiments of the present disclosure. IV catheter device 100 includes a catheter adapter 110 from which a catheter 111 extends distally. Although not shown, a needle assembly may oftentimes be secured to catheter adapter 110 and may be employed to insert catheter 111 into a patient's vasculature and subsequently detached from catheter adapter 110. IV catheter device 110 also includes an adapter 114 that is connected to a side port 112 of catheter adapter 110 via extension tubing 113. Adapter 114 can provide a connector 116 (whether integrated or separate) by which a probe assembly 130 (or any other probe assembly encompassed herein) can be coupled to IV catheter device 100 and through which a probe 140 (see FIGS. 2A and 2B) of probe assembly 130 gains access to catheter 111. In some embodiments, probe assembly 130 could be integrated into adapter 114 as opposed to being selectively coupled to adapter 114.

A connector 115 may also be connected to adapter 114 via extension tubing 113. A pinch clamp 117 may be provided on extension tubing 113. A blood collection set 120 is shown as being coupled to connector 115 but is only one example of a device that may be connected to IV catheter device 100. In other examples, probe assembly 130 or another probe assembly could be coupled to or integrated into connector 115 as opposed to connector 116. It is reiterated, however, that IV catheter device 100 is merely an example of an IV catheter device with which a probe configured in accordance with embodiments of the present disclosure may be used.

Probe assembly 130 is shown as including a probe housing 131, which can house probe 140 at least when probe 140 is not extended through catheter 111, a connector 132 by which probe assembly 130 can be connected to IV catheter device 100 (or another IV catheter device) and a probe actuator 133 by which a clinician can move probe 140 relative to catheter 111 by sliding probe actuator 133 along the length of probe housing 131. Although not visible in FIG. 1, with probe actuator 133 in the depicted position, the distal end of probe 140 may be advanced to the distal end of catheter 111. As described in greater detail below, in some embodiments, a probe assembly may be configured to allow a probe to be advanced and withdrawn within catheter 111 and also rotated. For example, after the distal end of probe 140 has been advanced near, to or beyond the distal end of catheter 111, probe actuator 133 may be configured to rotate clockwise and/or counter clockwise relative to the probe housing 131 to thereby cause the distal end of probe 140 to rotate.

FIGS. 2A and 2B illustrate probe assembly 130 in isolation. In FIG. 2A, probe actuator 133 is at a proximal-most position which has caused the distal end of probe 140 to be positioned at connector 132. In some embodiments, FIG. 2A may represent the configuration of probe assembly 130 prior to being connected to an IV catheter device such as IV catheter device 100. In contrast, in FIG. 2B, probe actuator 133 is at a distal-most position which has caused probe 140 to be advanced distally out from connector 132. A length of probe 140 and/or the configuration of probe actuator 133 can cause the distal end of probe 140 to be positioned proximate to (e.g., proximal to, at or distal to) the distal opening of catheter 111 (or the distal opening of the catheter of any other IV catheter device with which probe assembly 130 is compatible). The depicted configuration of probe assembly 130 is intended to be an example only. A probe assembly in accordance with embodiments of the present disclosure could have any suitable connector, any suitable probe housing and any suitable probe actuator.

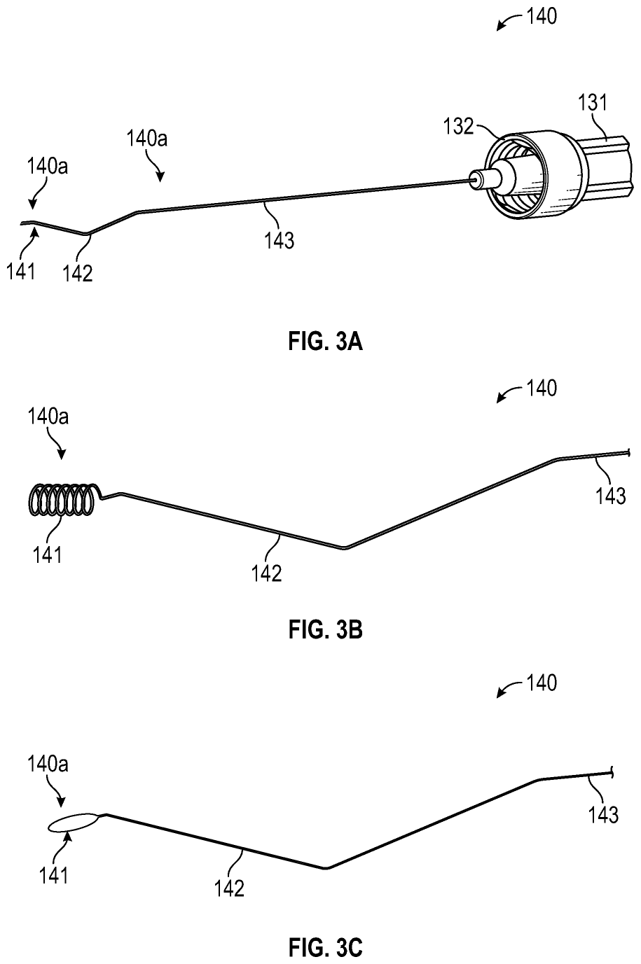

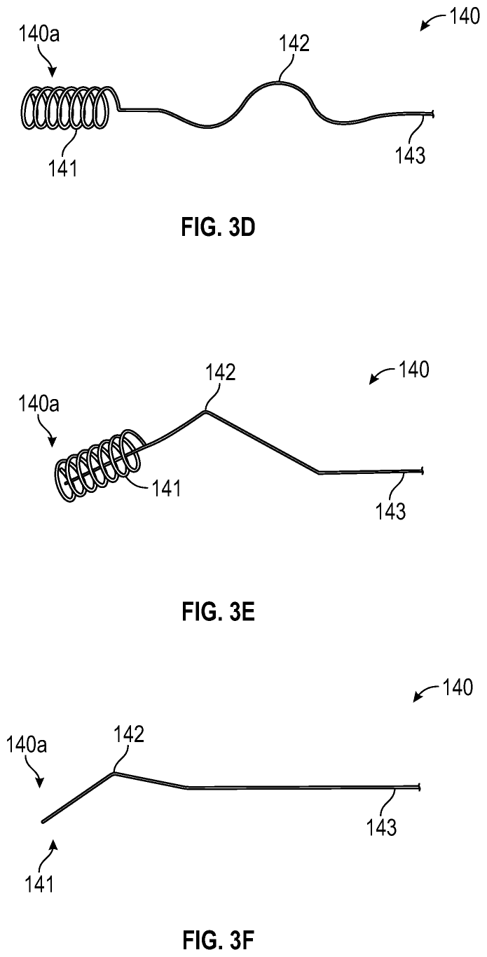

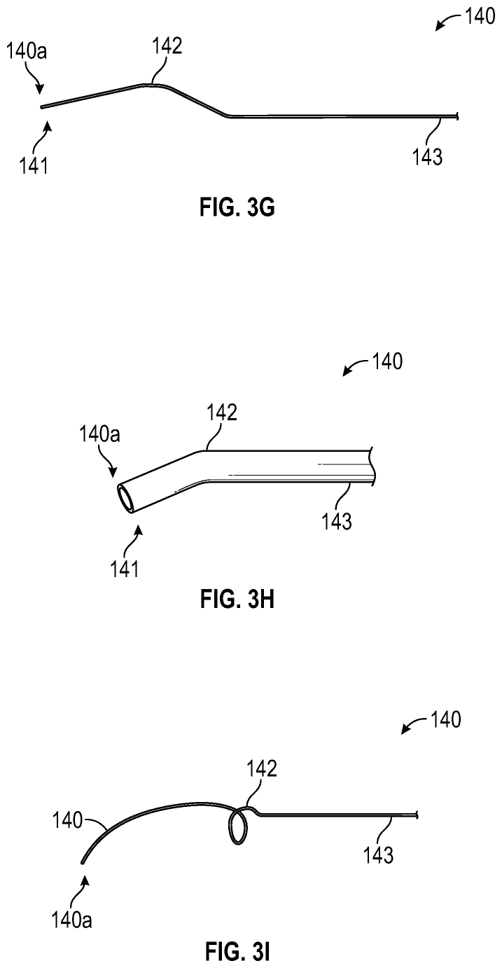

In accordance with embodiments of the present disclosure, probe 140 can be configured to reposition the distal end of catheter 111 when probe 140 is advanced into and/or rotated within catheter 111. FIGS. 3A-3I provide various examples of how probe 140 may be configured to cause this repositioning. FIG. 3A depicts a portion of probe 140 extending from a portion of probe housing 131 which employs a differently configured connector 132. FIG. 3A can therefore represent that probe 140 may be employed with many different variations of probe assembly 130. FIGS. 3B-3H each depict only a distal length of probe 140.

In each of FIGS. 3A-3I, probe 140 is shown as having a distal end 140a, a distal portion 141, a shaped portion 142 and a proximal portion 143. Shaped portion 142 should be construed as a length of probe 140 positioned at or towards distal end 140a that has a shape that deviates from a longitudinal axis of proximal portion 143 and that generally retains this shape when distal end 140a is positioned proximate to, at or extends distally out from a distal opening of catheter 111. It is noted that shaped portion 142 may flex, flatten or otherwise adapt its shape as it is advanced through catheter 111 due to the proximal lengths of catheter 111 being confined within the skin. However, when positioned at or near the distal end of catheter 111, shaped portion 142 may generally retain its shape to thereby act on the distal end of catheter 111 causing it to be repositioned. In some embodiments, shaped portion 142 may be separate from distal portion 141, while in other embodiments, shaped portion 142 may encompass distal portion 141 and possibly distal end 140a. As stated above, the shape of probe 140 would necessarily need to adapt to the confines of catheter 111 as probe 140 is advanced (e.g., the s-shape of catheter 111 as it transitions through the patient's skin and into the vasculature). Yet, even with such adaptations, shaped portion 142 is configured to substantially retain its shape relative to distal portion 141 and proximal portion 143 as will become apparent below.

FIGS. 3A-3I provide various examples of how shaped portion 142 may be configured. Embodiments of the present disclosure should not be limited to these examples. In particular, shaped portion 142 could be configured in many different ways which cause it to deviate from the longitudinal axis of proximal portion 143. For example, shaped portion 142 may comprise one or more lengths of probe 140 that deviate from the longitudinal axis including multiple lengths that are each at a different angle from one another and/or that lie in different planes. Similarly, shaped portion 142 may comprise one or more lengths of probe 140 that are curved relative to the longitudinal axis.

In FIG. 3A, shaped portion 142 is in the form of a v-shaped length of probe 140 that extends between proximal portion 143 and distal portion 141. In this example, distal portion 141 may extend along the same longitudinal axis as proximal portion 143 such that only shaped portion 142 deviates from the longitudinal axis. However, in some embodiments, distal portion 141 could extend along a different and non-parallel axis (e.g., by being upwardly oriented or downwardly oriented relative to proximal portion 143) and/or could extend along a different but parallel axis as the longitudinal axis (e.g., upwardly or downwardly offset relative to proximal portion 143). The configuration of probe 140 depicted in FIG. 3A is intended to apply a lifting force to the distal end of catheter 111 when distal portion 141 is at or beyond the distal opening of catheter 111 while shaped portion 142 remains at least partially within catheter 111. In some embodiments, the direction of this lifting force could be altered by rotating probe 140 relative to the position shown in FIG. 3A (e.g., to move the distal end of catheter 111 downward or side-to-side).

FIG. 3B provides an example similar to FIG. 3A except that distal portion 141 and distal end 140a are in the form of a coil. This coil may enhance probe 140's ability to remove an occlusion or an obstruction away from the distal opening of catheter 111 while facilitating fluid flow into or out from catheter 111 while probe 140 is positioned therein thereby increasing the patency of catheter 111.

FIG. 3C provides an example similar to FIG. 3B except that distal portion 141 and distal end 140a have an expanded and solid cross-sectional area. As with the coil, this solid, expanded area may enhance probe 140's ability to remove an occlusion or obstruction. Similar to what is shown in FIG. 3C, distal end 140a could be configured as a dome that is formed from or in some manner attached to distal portion 141 (e.g., via welding or an adhesive). In embodiments where distal portion 141 forms an expanded cross-sectional area (or larger outside diameter), this area may be tapered proximally to facilitate withdrawing distal portion 141 back into catheter 111 without damaging the distal opening or tip of catheter 111. Similarly, distal end 140a may be tapered distally to minimize damage to the vasculature as distal end 140a is advanced out of catheter 111.

FIG. 3D provides an example where shaped portion 142 has a shape that generally resembles a curved w and distal portion 141 and distal end 140a are in the form of a coil.

FIG. 3E provides an example where shaped portion 142 has an inverted v shape that encompasses distal portion 141. Also, distal portion 141 forms a coil.

FIG. 3F provides another example where shaped portion 142 has an inverted v shape that encompasses distal portion 141. However, the proximal length of shaped portion 142 deviates for the longitudinal axis of proximal portion 143 to a lesser extent than the distal length of shaped portion 142. Also, distal end 140a is positioned below the longitudinal axis of proximal portion 143.

FIG. 3G provides another example where shaped portion 142 has an inverted v shape that encompasses distal portion 141. However, the distal length of shaped portion 142 deviates for the longitudinal axis of proximal portion 143 to a lesser extent than the proximal length of shaped portion 142.

FIG. 3H provides an example where shaped portion 142 encompasses distal portion 141 and is in the form of a bend relative to proximal portion 143. Also, FIG. 3H represents that, in some embodiments, probe 140 could be in the form of a tube as opposed to a wire.

FIG. 3I provides an example where shaped portion 142 is in the form of a spiral 142 and distal portion 141 is curved relative to proximal portion 143.

The variations shown in FIGS. 3A-3I are not mutually exclusive and many such variations could be employed together. For example, distal portion 141 could form a coil in any of the described embodiments. Likewise, probe 140 could be formed of tubing in any of the described embodiments. Additionally, the orientation and lengths of the different parts of shaped portion 142 could be altered while the orientation of shaped portion 142 relative to distal portion 141 and/or proximal portion 143 could also be altered. In short, embodiments of the present disclosure should not be limited to the specific examples shown in the figures.

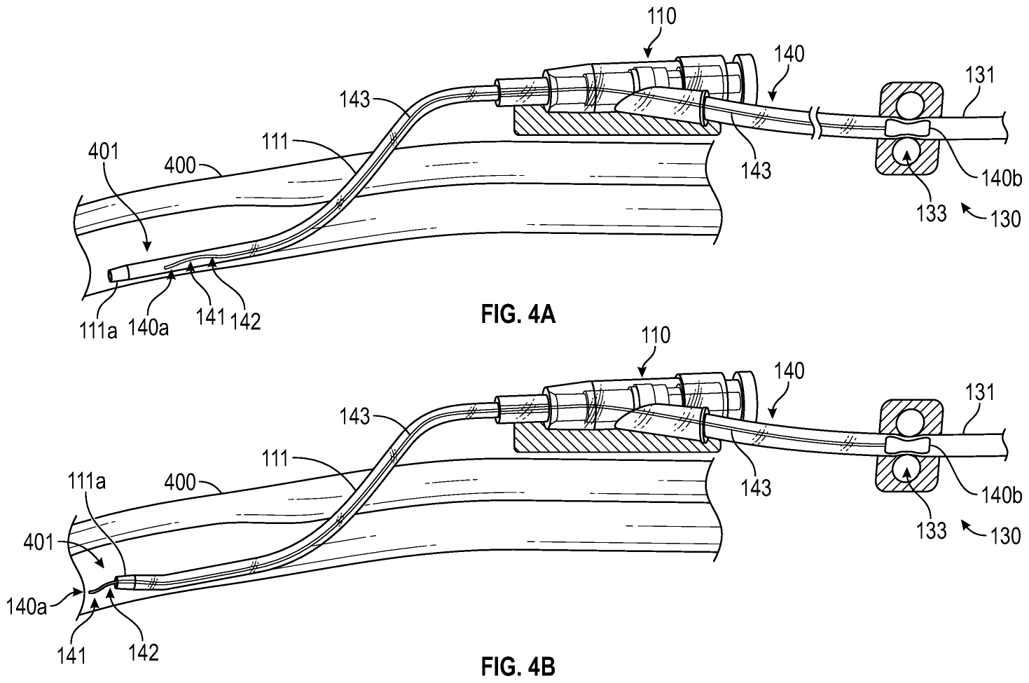

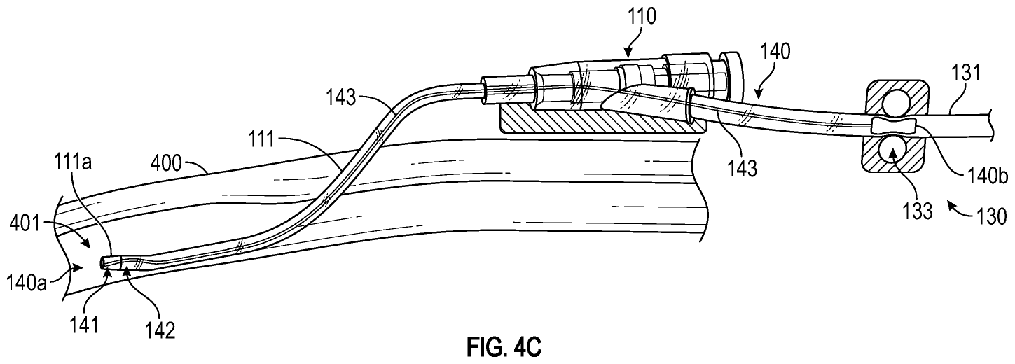

FIGS. 4A-4C provide an example of how probe 140 can cause the distal end of catheter 111 to be repositioned. FIG. 4A shows that catheter adapter 110 is resting on a patient's skin 400 while catheter 111 is inserted into the patient's vasculature 401. FIG. 4A also shows that probe assembly 130 has been connected to catheter adapter 110 and probe 140 has been partially advanced into catheter 111. In FIGS. 4A-4C, probe 140 resembles the example shown in FIG. 3G. Proximal portion 143 is shown as adapting to the s-shape of catheter 111 while shaped portion 142 and distal portion 141 substantially maintain their shape within catheter 111. In FIG. 4A, because shaped portion 142 is spaced substantially away from distal end 111a of catheter 111, distal end 111a remains in its natural position against the wall of vasculature 401. In some embodiments, however, shaped portion 142 may be configured to apply a lifting force on distal end 111a before distal portion 141 reaches or extends distally beyond distal end 111a.

Turning to FIG. 4B, it is now assumed that a clinician has employed probe actuator 133 to further slide probe 140 into catheter 111. For example, probe actuator 133 could interface with a proximal end 140b of probe 140 so that probe 140 slides and/or rotates as probe actuator 133 is slid or rotated. In the depicted example, proximal end 140b is in the form of a wedge on which probe actuator 133 acts through the sidewall of probe housing 131. In such embodiments, probe housing 131 may be in the form of extension tubing.

Due to distal movement of probe actuator 133, distal end 140a of probe 140 now extends out from distal end 111a of catheter 111. Due to shaped portion 142 being positioned at distal end 111a, shaped portion 142 will apply a lifting force against distal end 111a thereby causing distal end 111a to be lifted away from the wall of vasculature 401. More particularly, because shaped portion 142 substantially retains its shape within catheter 111, the inverted v shape of shaped portion 142 relative to proximal portion 143 causes distal end 111a to be upwardly oriented relative to proximal portions of catheter 111. This lifting not only moves distal end 111a away from the wall of vasculature 401 but also pivots distal end 111a. In this way, if distal end 111a had become positioned against the wall of the vasculature or other structure or had become otherwise occluded, the repositioning of catheter 111 may regain the ability to collect a blood sample or inject a fluid through catheter 111. In this context, lifting is used relatively and could encompass downward or sideward movement of distal end 111a depending on the rotational orientation of probe 140. FIG. 4C represents that shaped portion 142 may cause lifting force to be applied against distal end 111a of catheter 111 in a similar manner even when distal end 140a of probe 140 is at or near distal end 111a but does not extend distally out from distal end 111a.

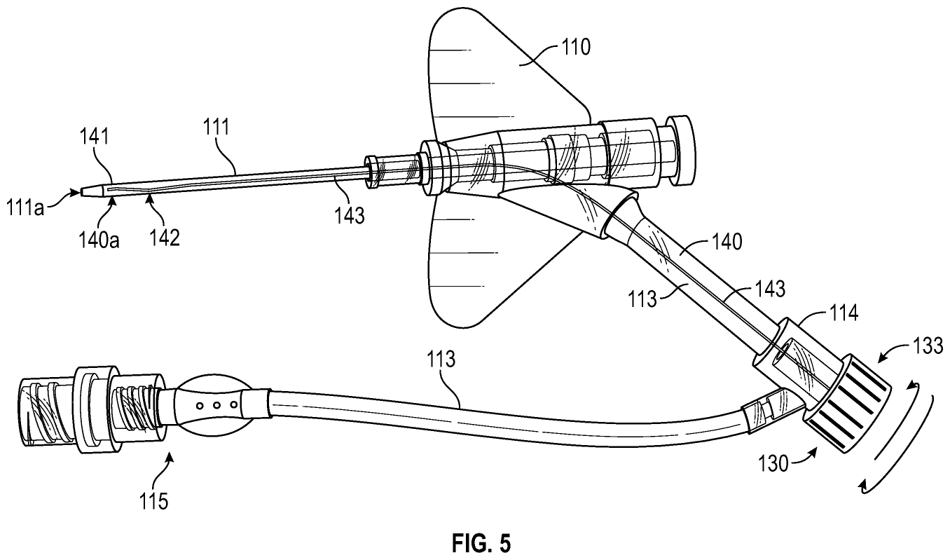

FIG. 5 illustrates an example where probe 140 is configured to rotate relative to catheter 111. In this example, probe assembly 130 is configured to enable a clinician to rotate probe 140 by rotating probe actuator 133. For example, with reference to FIGS. 4A and 4B, proximal end 140b of probe 140 could be coupled, whether directly or indirectly, to probe actuator 133 so that the entirety of probe 140 is rotated when the clinician rotates probe actuator 133. By rotating probe 140, the clinician can adjust the orientation of shaped portion 142 to alter in which direction distal end 111a of catheter 111 is repositioned. For example, the clinician could rotate probe 140 to cause distal end 111a to travel along a circular path, pivot back and forth, or otherwise move until finding a position in which blood flow is unimpeded.

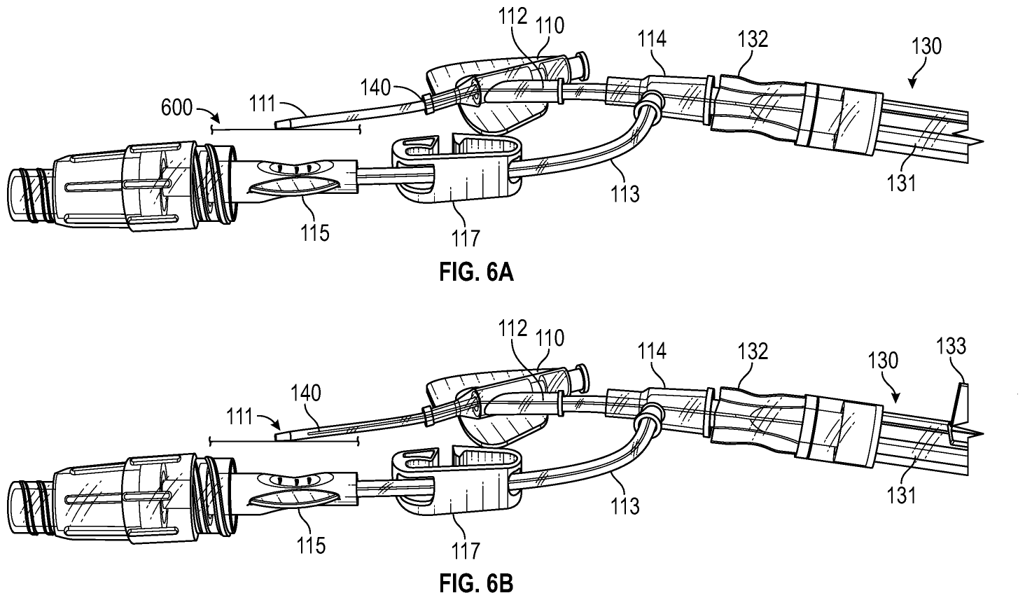

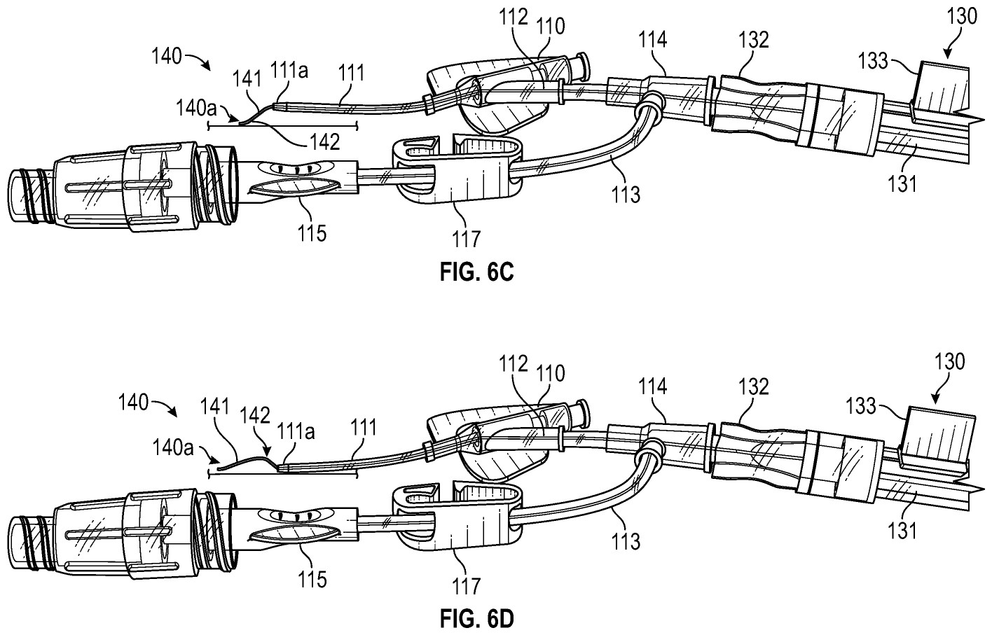

FIGS. 6A through 6D provide another example of how catheter 111 can be repositioned using probe 140. In these figures, catheter 111 is depicted relative to a surface 600 which could represent the vein wall in some embodiments. In FIG. 6A, probe 140 has not yet been advanced into catheter 111. Accordingly, catheter 111 is resting on the surface. In FIG. 6B, probe actuator 133 has been advanced toward adapter 114 but probe 140 has not yet reached distal end 111a of catheter 111. Accordingly, catheter 111 remains resting on the surface.

Turning to FIG. 6C, probe actuator 133 has been further advanced thereby causing distal end 140a of probe 140 to extend beyond distal end 111a of catheter 111. In this example, shaped portion 142 resembles the example of FIG. 3F. Accordingly, as distal portion 141 extends out from distal end 111a, distal end 140a may contact the surface (which could represent a patient's vasculature). Due to shaped portion 142, distal end 111a will be lifted upwardly away from the surface. More particularly, because shaped portion 142 retains its shape, catheter 111 will conform to the shape of shaped portion 142.

Turning to FIG. 6D, probe actuator 133 has been advanced to its distal-most position which in turn has caused shaped portion 142 to be extended fully out from distal end 111a. As a result, distal end 111a is no longer being lifted by shaped portion 142 and has therefore returned to rest on the surface. It is noted that, in some embodiments, probe 140 may not be configured to allow shaped portion 142 to extend completely out from distal end 111a. Accordingly, embodiments of the present disclosure should encompass cases where probe assembly 130 is designed to prevent shaped portion 142 from extending out from distal end 111a and cases where probe assembly 130 is designed to allow shaped portion 142 to extend partially or completely out from distal end 111a.

With reference to FIG. 6C, if probe 140 were rotated, distal end 111a of catheter 111 could be caused to travel along a circular path. Also, if probe 140 included a coil or other enlarged structure at distal end 140a, trauma to a patient's vasculature could be minimized while also potentially enhancing the removal of any occlusion.

In any case, by employing probe 140 with shaped portion 142, catheter 111 can be repositioned to enable blood collection or fluid injection through catheter 111 even when catheter 111 has become occluded. As such, probe 140 may prolong the patency of a catheter without requiring the use of a device that provides a separate fluid pathway from the catheter. Because probe 140 enables catheter 111 to be used to collect a blood sample, an improved flow rate may exist during the collection of the blood sample relative to the flow rate that would exist when employing a separate, smaller tube inserted through catheter 111. However, as is represented by FIG. 3H, a probe that provides a shaped portion 142 could be in the form of a tube that provides a separate fluid pathway.

Probe 140 may be formed of any suitable material including, for example, metals such as nitinol or stainless steel, polymers such as nylon, polytetrafluoroethylene (PTFE) or polyetherimide or combinations of such materials. In some embodiments, probe 140 may be formed of a first material with a coating of a second material such as, for example, a stainless steel or nitinol core with a nickel coating or a metal core with a polymer coating.

In some embodiments, distal end 140a of probe 140 may be rounded and/or tapered to cause probe 140 to be more atraumatic when it is advanced distally beyond catheter 111. Also, a tapered distal end 140a may facilitate withdrawing probe 140 back into catheter 111 while minimizing damage to distal end 111a of catheter 111.

In some embodiments, in addition to providing the ability to reposition the distal end 111a of catheter 111, probe 140 can also reinforce proximal portions of catheter 111. For example, probe 140 may be formed of a material that is more rigid or resilient than the material from which catheter 111 is formed. Therefore, with probe positioned inside catheter 111, proximal portion 143 of probe 140 can prevent kinking in catheter 111 (e.g., in the s-shaped portion of catheter 111).

In some embodiments, probe 140 may also function to reposition distal end 111a of catheter 111 by altering the s-shape of catheter 111. For example, due to the more rigid material from which probe 140 may be formed, proximal portion 143 may straighten the s-shaped region of catheter 111 which may cause distal end 111a of catheter 111 to be advanced within the vasculature. On the other hand, shaped portion 142 could be positioned within the s-shaped portion of catheter 111 and may cause it to curve more (i.e., to form a tighter s shape) which may cause distal end 111a of catheter 111 to retract within the vasculature.

In summary, a probe can be configured with a shaped portion that can lift, advance, retract, and/or swivel a distal end of a catheter to thereby reposition the catheter within a patient's vasculature. This repositioning can move the catheter relative to the wall or other anatomy of the vasculature and relative to any obstructions such as a thrombus that may have formed. By repositioning the catheter, the probe prolongs the patency of the catheter including facilitating the collection of a blood sample through a long-dwelling catheter.

All examples and conditional language recited herein are intended for pedagogical objects to aid the reader in understanding the present disclosure and the concepts contributed by the inventor to furthering the art and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

What is claimed:

1. An intravenous catheter device comprising:
a catheter adapter;
a catheter that extends distally from the catheter adapter; and
a probe assembly that couples to the catheter adapter, the probe assembly including a probe that selectively extends into the catheter, the probe having a shaped portion for causing a distal end of the catheter to be repositioned as the probe is selectively extended into the catheter.

2. The intravenous catheter device of claim 1, wherein the probe assembly includes a probe actuator by which the probe is selectively extended into the catheter.

3. The intravenous catheter device of claim 2, wherein the probe actuator is configured to advance axially, rotate or both advance axially and rotate to thereby cause the probe to advance axially, rotate or both advance axially and rotate within the catheter.

4. The intravenous catheter device of claim 1, wherein the probe assembly is one of integrated into or selectively coupled to the catheter adapter.

5. The intravenous catheter device of claim 1, wherein the probe has a proximal portion and a distal portion, and wherein the shaped portion is positioned between the proximal portion and the distal portion.

6. The intravenous catheter device of claim 5, wherein the shaped portion encompasses the distal portion of the probe.

7. The intravenous catheter device of claim 5, wherein the distal portion forms a coil or an expanded cross-sectional area.

8. The intravenous catheter device of claim 1, wherein the shaped portion has a v shape or a w shape.

9. The intravenous catheter device of claim 1, wherein the shaped portion is configured to cause the distal end of the catheter to be repositioned when a distal end of the probe is positioned proximal to, at or distal to the distal end of the catheter.

10. The intravenous catheter device of claim 1, wherein the shaped portion comprises a spiral.

11. The intravenous catheter device of claim 1, wherein the probe includes a proximal portion and the shaped portion is distal to the proximal portion, and wherein the shaped portion includes a first length that deviates from a longitudinal axis of the proximal portion by a first angle and a second length that deviates from the longitudinal axis of the proximal portion by a second angle different from the first angle.

12. The intravenous catheter device of claim 1, wherein the probe is one of a wire or a tube.

13. The intravenous catheter device of claim 1, wherein the probe assembly includes a probe housing within which the probe is housed and a probe actuator positioned at least partially outside of the probe housing, the probe actuator interfacing with the probe to cause the probe to be selectively advanced out from the probe housing and into the catheter.

14. The intravenous catheter device of claim 13, wherein the probe actuator also interfaces with the probe to cause the probe to rotate within the catheter.

15. A probe assembly for use with a catheter of an intravenous catheter device, the probe comprising:
a probe housing;
a probe actuator coupled to the probe housing; and
a probe that is housed within the probe housing, the probe actuator being configured to selectively advance the probe from the probe housing and into the catheter of the intravenous catheter device, wherein the probe comprises:
a proximal portion;
a distal end; and
a shaped portion positioned between the proximal portion and the distal end, the shaped portion being configured to reposition a distal end of the catheter as the probe is selectively advanced within the catheter.

16. The probe assembly of claim 15, wherein the shaped portion includes a first length that deviates from a longitudinal axis of the proximal portion by a first angle and a second length that deviates from the longitudinal axis of the proximal portion by a second angle different from the first angle.

17. The probe assembly of claim 15, wherein the probe actuator is configured to selectively rotate the probe within the catheter.

18. The probe assembly of claim 15, wherein the shaped portion of the probe encompasses the distal end of the probe.

19. A method of repositioning a distal end of a catheter within a patient's vasculature, the method comprising:
providing an intravenous catheter device having a catheter adapter, a catheter that extends distally from the catheter adapter and a probe assembly that couples to the catheter adapter, the probe assembly including a probe that selectively extends into the catheter, the probe having a shaped portion.
inserting the catheter into a patient's vasculature; and
while the catheter is inserted into the patient's vasculature, advancing the probe within the catheter to cause the shaped portion of the probe to be positioned proximate to a distal end of the catheter, the shaped portion causing the distal end of the catheter to be repositioned.

20. The method of claim 19, further comprising:
advancing the probe to cause a distal end of the probe to extend distally beyond the distal end of the catheter, the distal end of the probe being configured to cause an occlusion or obstruction to be removed from the distal end of the catheter.
